(19) United States
(12) Patent Application Publication
(10) Pub. No.: US 2013/0110045 A1
(43) Pub. Date: May 2, 2013

(54) SINGLE USE INTRAVENOUS THERAPY ADMINISTERING DEVICE WITH NEEDLE SAFETY COVERS

(71) Applicant: Ming-Yuan WU, Taipei City (TW)

(72) Inventor: Ming-Yuan WU, Taipei City (TW)

(21) Appl. No.: 13/285,809

(22) Filed: Oct. 31, 2011

(51) Int. Cl. A61M 5/50

(52) U.S. Cl. 604/111

(57) ABSTRACT

A single use intravenous (IV) therapy administering device comprising an IV bag puncture needle assembly, a drip chamber, a flow regulator, a fluid filter, an IV needle assembly is disclosed. An IV infusion line provides a conduit to allow medical fluid to flow from the IV bag puncture needle assembly to the IV needle assembly. A used indicator line provides a conduit to allow colored fluid to flow from the IV bag puncture needle assembly to the fluid filter. Used indicators on the IV bag puncture needle assembly and the IV needle assembly and the colored fluid in the used indicator line allow the administering device to be identified if the device has been used. Retractable safety covers on the IV bag puncture needle assembly and the IV needle assembly prevent accidental exposure to the needles.

### BACKGROUND OF THE INVENTION

1. Field of the Invention

The present invention relates to medical devices. More specifically, the present invention discloses a safe and effective device for administering medicinal fluid intravenously that indicates when the device has been previously used and provides two needle safety covers that prevent accidental contact with the needles.

2. Description of the Prior Art

Needles are commonly used medical implements for injecting medicine into a patient. For example, a medical professional draws medicine into the syringe and then injects the medicine into a patient through a needle in the syringe or a needle is inserted into a patient's vein for administering an intravenous drip.

While useful, the conventional needle device has various disadvantages that permit potentially dangerous use of the needle. Misuse of the traditional needle device can be fatal or detrimental to a patient's health.

One major disadvantage of the conventional needle device is that the needle can easily be used again. The conventional needle or components of the device can easily be used again since the conventional device provides no inherent means to prevent reuse.

After a patient is injected with a medicine, residue such as blood, medicine, virus, bacteria, or other contaminants remain on the needle or inside the needle device. If another patient is injected using the used device, the second patient can be contaminated with residue from the first patient.

If the first patient is infected with a virus, the second patient is susceptible to contracting the virus from the previously used needle or device. The second patient is also susceptible if the second patient is allergic to the medicine used with the first patient.

Additionally, it can be difficult to determine whether or not the device has been previously used.

Therefore, there is need for an improved medical device that effectively delivers medicine intravenously to a patient while automatically preventing reuse of the device.

### SUMMARY OF THE INVENTION

To achieve these and other advantages and in order to overcome the disadvantages of the conventional method in accordance with the purpose of the invention as embodied and broadly described herein, the present invention provides a medical device for use with intravenous therapy that visually indicates when the medical device has been previously used or is currently in use.

The single use intravenous (IV) therapy administering device of the present invention further comprises a safety cover that slides down to expose a puncture needle and slides up to cover the puncture needle after use. This safety cover protects medical personal from accidental contact with the puncture needle.

The medical device for use during intravenous therapy of the present invention comprises an intravenous (IV) bag puncture needle assembly, a drip chamber, a flow regulator, a medical fluid filter, and an intravenous (IV) needle assembly, an infusion line, and a used indicator line.

The IV bag puncture needle assembly comprises a puncture needle disposed in a base. In use, the puncture needle is inserted into an IV bag containing medical fluid or medicine. The fluid contents of the IV bag begins flowing through the hollow puncture needle and into an IV fluid reservoir inside the base. The IV fluid continues flowing into the infusion line attached to the base of the IV bag puncture needle assembly.

The IV bag puncture needle assembly further comprises a safety cover that slides down on the base to expose the puncture needle and slides up to cover the puncture needle after use. The safety cover further comprises a bladder puncture needle disposed in a cover arm of the safety cover. When the safety cover is pulled back to expose the puncture needle, the bladder puncture needle penetrates a bladder disposed in the base. The bladder contains a colored fluid. When the bladder puncture needle has punctured the bladder, the colored fluid begins leaking out and flows into the used indicator line connected to the base of the IV bag puncture needle assembly.

The fluid in the IV bag flows through the IV puncture needle assembly, through the drip chamber, through the fluid filter, through the IV needle assembly, and into a patient's vein via the IV infusion line connecting the components.

The colored fluid from the punctured bladder flows from the IV puncture needle assembly, along side the drip chamber, and along side the fluid filter via the used indicator line connecting the components and stops at a used line end stop or a used line end plug.

The colored fluid that has flowed through the used indicator line clearly identifies the IV therapy administering device as previously used or currently in use. This prevents accidental re-use of the device and protects patient's from contamination and bacterial infections from re-used devices.

These and other objectives of the present invention will become obvious to those of ordinary skill in the art after reading the following detailed description of preferred embodiments.

It is to be understood that both the foregoing general description and the following detailed description are exemplary, and are intended to provide further explanation of the invention as claimed.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention. In the drawings:

FIGS. 1A-1B are drawings illustrating a single use intravenous therapy administering device with needle safety covers according to embodiments of the present invention;

FIG. 2A is a side view drawing illustrating an intravenous bag puncture needle assembly of a single use intravenous therapy administering device with needle safety covers according to an embodiment of the present invention;

FIG. 2B is a front view drawing illustrating an intravenous bag puncture needle assembly of a single use intravenous therapy administering device with needle safety covers according to an embodiment of the present invention;

FIG. 2C is a cross-sectional view drawing illustrating an intravenous bag puncture needle assembly of a single use intravenous therapy administering device with needle safety covers according to an embodiment of the present invention;

FIG. 2D is a top view drawing illustrating an intravenous bag puncture needle assembly of a single use intravenous therapy administering device with needle safety covers according to an embodiment of the present invention;

FIGS. 2E-2G are cross-sectional view drawings illustrating an intravenous bag puncture needle assembly of a single use intravenous therapy administering device with needle safety covers according to embodiments of the present invention;

FIGS. 2H-2J are front view drawings illustrating an intravenous bag puncture needle assembly of a single use intravenous therapy administering device with needle safety covers according to embodiments of the present invention;

FIGS. 2K-2N are cross-sectional drawings illustrating an intravenous bag puncture needle assembly of a single use intravenous therapy administering device with needle safety covers according to embodiments of the present invention;

FIG. 2O is a front view drawing illustrating an intravenous bag puncture needle assembly of a single use intravenous therapy administering device with needle safety covers according to an embodiment of the present invention;

FIG. 2P is a cross-sectional view drawing illustrating an intravenous bag puncture needle assembly of a single use intravenous therapy administering device with needle safety covers according to an embodiment of the present invention;

FIG. 3A is a side view drawing illustrating a drip chamber of a single use intravenous therapy administering device with needle safety covers according to an embodiment of the present invention;

FIG. 3B is a front view drawing illustrating a drip chamber of a single use intravenous therapy administering device with needle safety covers according to an embodiment of the present invention;

FIG. 3C is a cross-sectional view drawing illustrating a drip chamber of a single use intravenous therapy administering device with needle safety covers according to an embodiment of the present invention;

FIG. 3D is a top view drawing illustrating a drip chamber of a single use intravenous therapy administering device with needle safety covers according to an embodiment of the present invention;

FIG. 4A is a side view drawing illustrating a fluid filter of a single use intravenous therapy administering device with needle safety covers according to an embodiment of the present invention;

FIG. 4B is a front view drawing illustrating a fluid filter of a single use intravenous therapy administering device with needle safety covers according to an embodiment of the present invention;

FIG. 4C is a cross-sectional view drawing illustrating a fluid filter of a single use intravenous therapy administering device with needle safety covers according to an embodiment of the present invention;

FIG. 4D is a top view drawing illustrating a fluid filter of a single use intravenous therapy administering device with needle safety covers according to an embodiment of the present invention;

FIG. 5A is a side view drawing illustrating an intravenous needle assembly of a single use intravenous therapy administering device with needle safety covers according to an embodiment of the present invention;

FIG. 5B is a cross-sectional view drawing illustrating an intravenous needle assembly of a single use intravenous therapy administering device with needle safety covers according to an embodiment of the present invention;

FIG. 5C is a side view drawing illustrating an intravenous needle assembly of a single use intravenous therapy administering device with needle safety covers according to an embodiment of the present invention;

FIG. 5D is a top view drawing illustrating an intravenous needle assembly of a single use intravenous therapy administering device with needle safety covers according to an embodiment of the present invention;

FIGS. 5E-5F are cross-sectional view drawings illustrating an intravenous needle assembly of a single use intravenous therapy administering device with needle safety covers according to embodiments of the present invention;

FIG. 5G is a side view drawing illustrating an intravenous needle assembly of a single use intravenous therapy administering device with needle safety covers according to an embodiment of the present invention;

FIG. 5H is a side view drawing illustrating an intravenous needle assembly of a single use intravenous therapy administering device with needle safety covers according to an embodiment of the present invention;

FIG. 5I is a cross-sectional view drawing illustrating an intravenous needle assembly of a single use intravenous therapy administering device with needle safety covers according to an embodiment of the present invention;

FIG. 5J is a side view drawing illustrating an intravenous needle assembly of a single use intravenous therapy administering device with needle safety covers according to an embodiment of the present invention; and

FIGS. 6A-6C are drawings illustrating a flow regulator of a single use intravenous therapy administering device with needle safety covers according to an embodiment of the present invention.

### DESCRIPTION OF THE PREFERRED EMBODIMENTS

Reference will now be made in detail to the preferred embodiments of the present invention, examples of which are illustrated in the accompanying drawings. Wherever possible, the same reference numbers are used in the drawings and the description to refer to the same or like parts.

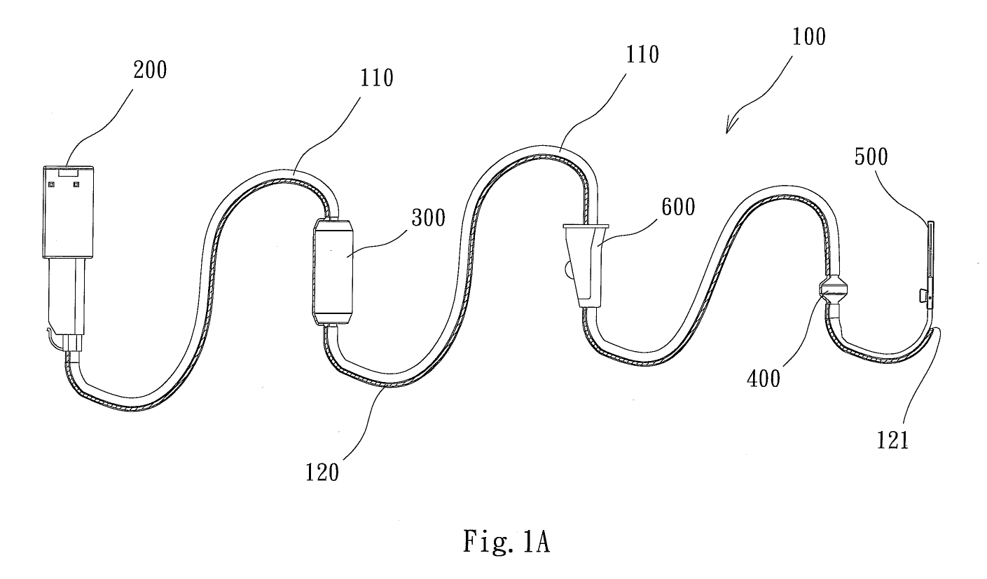

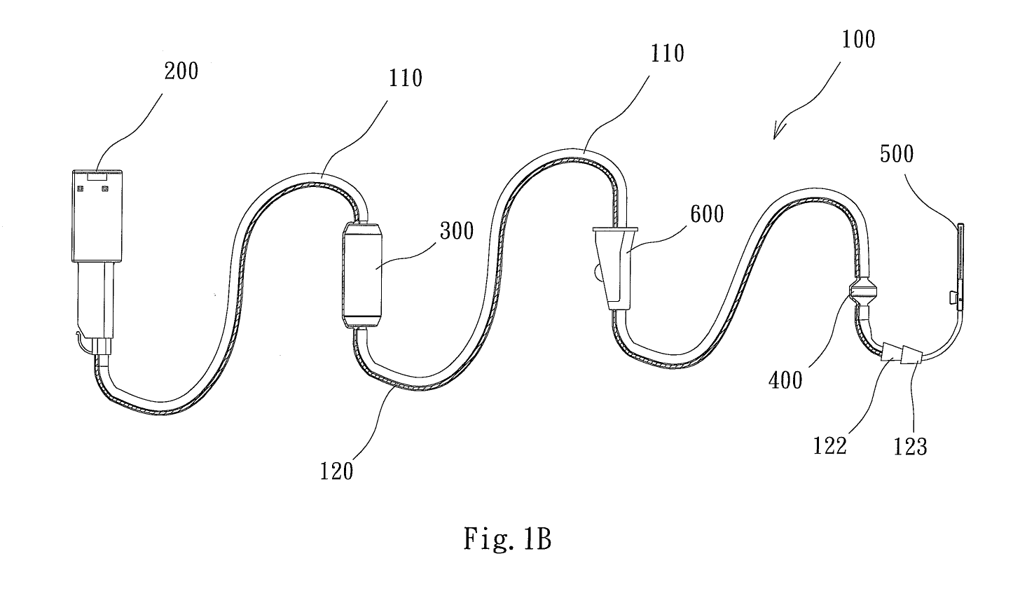

Refer to FIG. 1A. The single use IV therapy administering device with puncture needle safety cover 100 according to an embodiment of the present invention comprises an IV bag puncture needle assembly 200, a drip chamber 300, a flow regulator 600, a fluid filter 400, an IV needle assembly 500, an IV infusion line 110, and a used indicator line 120.

A needle of the IV bag puncture needle assembly 200 is inserted into a bag containing an IV fluid such as, for example, blood-based products, blood substitutes, medications, volume expanders, specialty pharmaceuticals, or other medical solutions or fluids. Once the needle is inserted into the IV bag, the fluid begins to flow through the needle and the base of the IV bag puncture needle assembly 200. Additionally, a colored fluid contained in a bladder inside the IV bag puncture needle assembly 200 begins to flow when the safety cover of the IV bag puncture needle assembly 200 is retracted.

The drip chamber 300 is connected to the IV bag puncture needle assembly 200 by a length of infusion line 110 and a used indicator line 120. The medical fluid flows from the IV bag puncture needle assembly 200 through the infusion line 110 and into the drip chamber 300. The drip chamber 300 allows for a small quantity of medical fluid to be temporarily stored in order to ensure that a continuous flow of medical fluid reaches the IV needle assembly 500. The use of the drip chamber 300 prevents air entering the blood stream and also allows for an estimation of flow rate.

Similarly, the drip chamber 300 is connected to the fluid filter 400 by a length of infusion line 110 and a used indicator line 120. Medical fluid flows out of the drip chamber 300 and into the fluid filter 400 through the infusion line 110.

Disposed between the drip chamber 300 and the fluid filter 400 is a flow regulator 600. The flow regulator 600 is adjustable to control the flow of the medical fluid.

The medical fluid flowing out of the fluid filter 400 passes through the IV needle assembly 500 and into a patient.

The colored fluid exiting the IV bag puncture needle assembly 200 flows through the used indicator line 120. The drip chamber 300 and the fluid filter 400 both comprise used lines that allow the colored fluid to flow around a side of the drip chamber 300 and the fluid filter 400. A used line end stop 121 blocks the colored fluid at the end of the used indicator line 120.

The colored fluid in the used indicator line 120 makes it simple and convenient to identify that the IV therapy administering device with puncture needle safety cover 100 has been used or is being used. The used indicator line 120 and the colored fluid ensure that the IV therapy administering device with puncture needle safety cover 100 will not accidentally be reused.

The IV infusion line 110 comprises, for example, a hollow tubing or hollow conduit.

In an embodiment of the present invention the used indicator line 120 comprises a hollow tubing or hollow conduit.

In another embodiment of the present invention the used indicator line 120 comprises a hollow tubing with an absorbent material disposed inside the hollow tubing. The absorbent material comprises, for example, an absorbent cord, thread, or fiber lining.

Refer to FIG. 1B, which is a drawing illustrating a single use intravenous therapy administering device with needle safety covers according to an embodiment of the present invention.

In the embodiment illustrated in FIG. 1B, the used indicator line 120 ends at a used line end plug 122. An IV line connector 123 connects to the used line end plug 122 and allows medical fluid to flow from the fluid filter 400 through the used line end plug 122, the IV line connector 123, and a short length of infusion line 110 and into the IV needle assembly 500.

In certain situations the IV needle remains in the patient's vein for several IV therapy treatments in order to prevent damage to the vein from repeated injecting a needle into the vein. The embodiment illustrated in FIG. 1B allows the IV needle assembly 500 to be separated from the rest of the IV therapy administering device 100 by disconnecting the IV line connector 123 from the used line end plug 122. After disconnecting the IV line connector 123 from the used line end plug 122, the IV needle assembly 500 and the IV line connector 123 with the short length of IV infusion line 110 remain with the patient and the remainder of the IV therapy administering device 100 can be discarded. At a later time a used line end plug 122 of a new IV therapy administering device 100 can easily be connected to the original IV line connector 123.

Additionally, since the connection of the IV line connector 123 and the used line end plug 122 is separated from the IV needle assembly 500 by the short length of infusion line 110, movement of the IV needle in the patient's vein is reduced and the patient's comfort is improved.

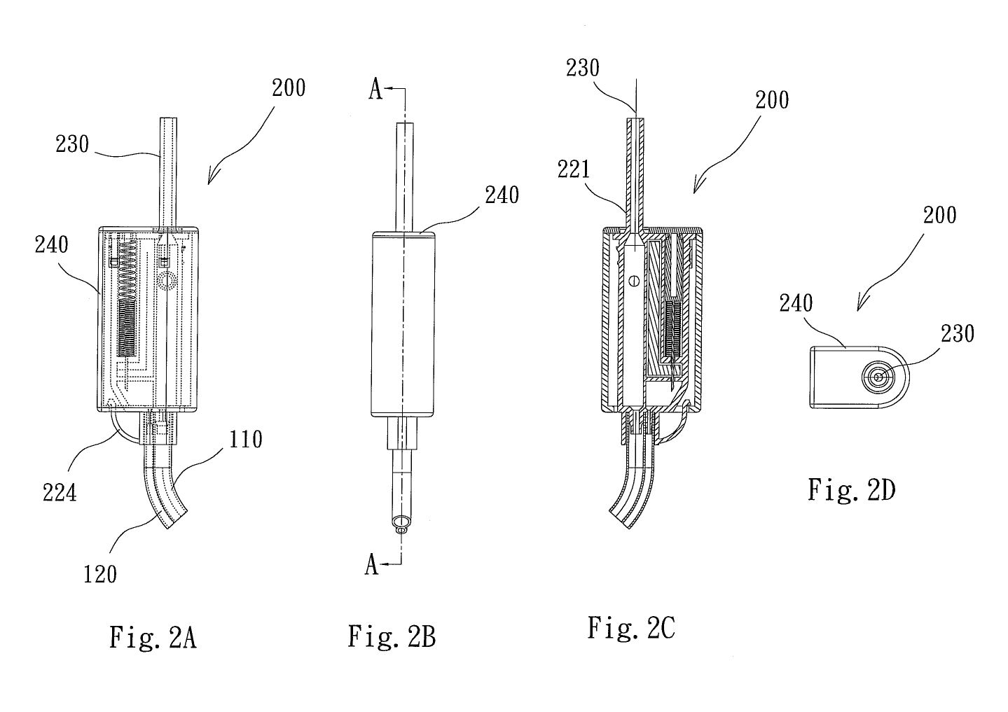

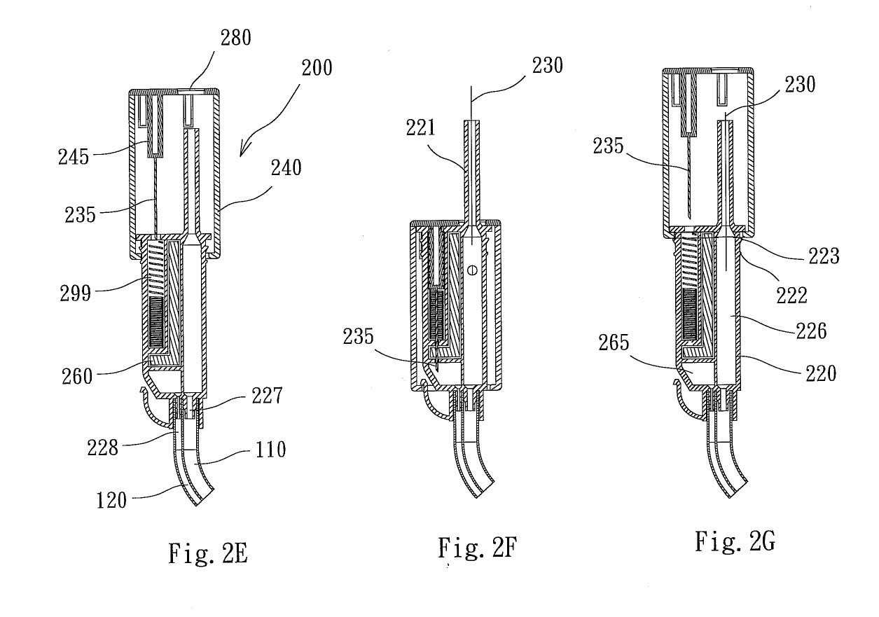

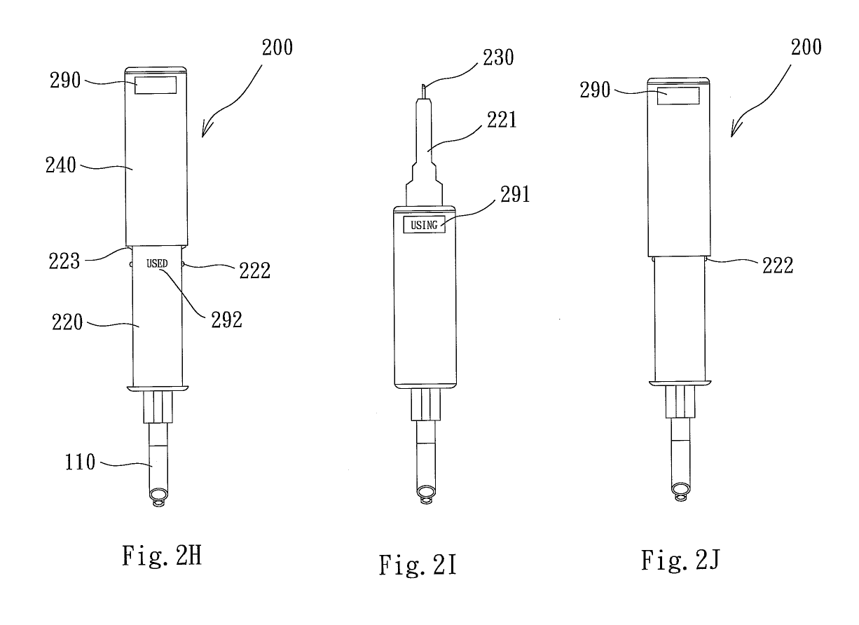

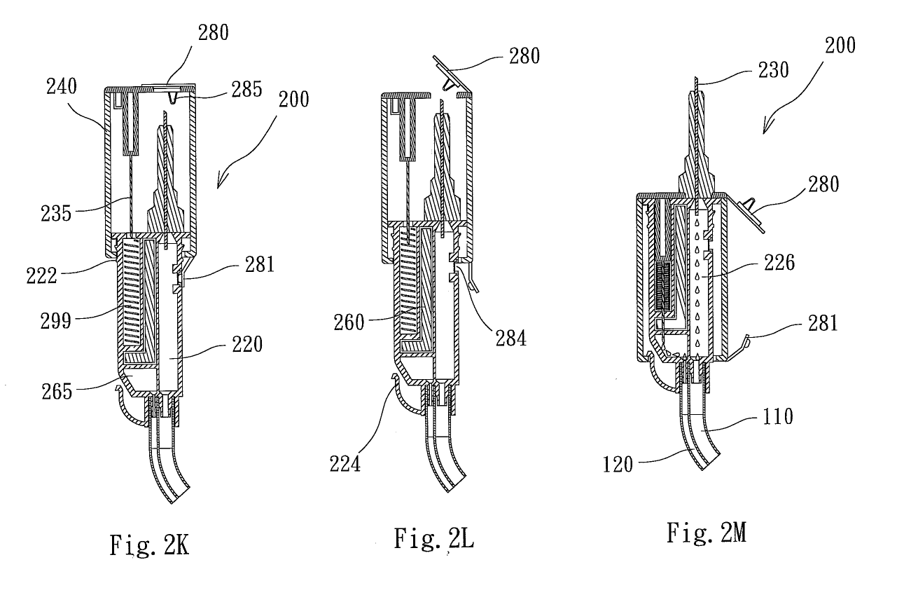

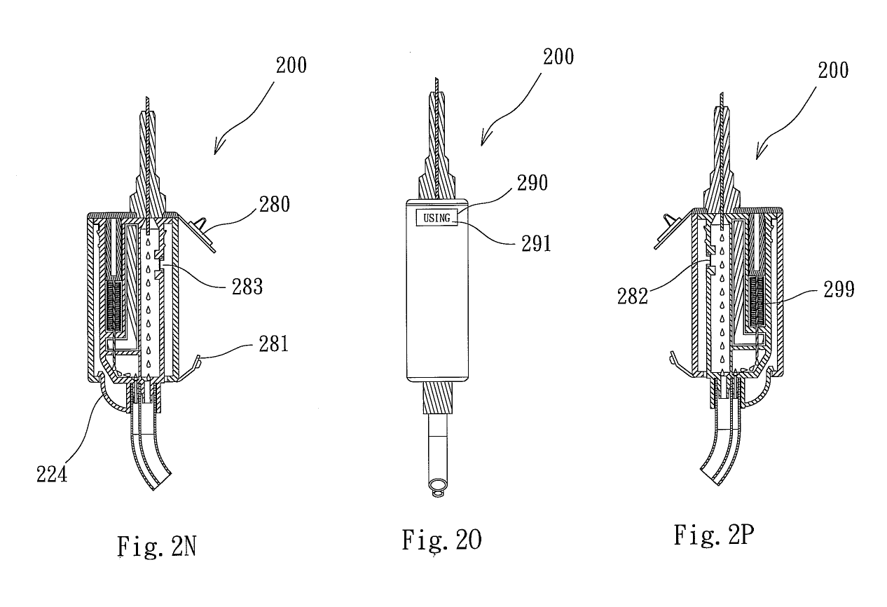

Refer to FIGS. 2A-2P, which are drawings illustrating an intravenous bag puncture needle assembly of a single use intravenous therapy administering device with needle safety covers according to an embodiment of the present invention.

The IV bag puncture needle assembly 200 allows fluid in an IV bag to flow through the IV bag puncture needle assembly 200 and into the IV infusion line 110. The IV bag puncture needle assembly 200 comprises a puncture assembly base 220, an IV bag puncture needle 230, and a puncture needle safety cover 240. The puncture needle safety cover 240 is moveably connected with the puncture assembly base 220. The puncture needle safety cover 240 surrounds the IV bag puncture needle 230 to prevent accidental exposure to the IV bag puncture needle 230. For use, the puncture needle safety cover 240 is retracted or moved backward on the puncture assembly base 220 to expose the IV bag puncture needle 230.

The puncture needle base 220 further comprises a base tip 221, a front holder 222, a front lock 223, a rear lock 224, an IV reservoir 226, a puncture needle IV outlet 227, a puncture needle used line outlet 228, and an air filter 284.

The base tip 221 extends from the puncture needle base 220 and around the IV bag puncture needle 230 leaving a length of the tip of the IV bag puncture needle 230 and a length of the rear of the IV bag puncture needle 230 exposed. When the IV puncture needle 230 is inserted into the IV bag base tip 221 enters the hole in the IV bag that the IV bag puncture needle 230 made when puncturing the IV bag.

The air filter 284 is disposed in a side of the puncture needle base 220. The air filter 284 comprises an air hole 283 and an air filter membrane 282. The air filter 284 allows air or pressure to pass through the air hole 283 and the air filter membrane 282 and enter the IV reservoir 226. The air filter 284 further comprises an air filter cap 281 for covering the air hole 283 when the air filter 284 is not required or not in use. Typically, the air filter cap 281 is removed, exposing the air hole 283, just prior to use.

The puncture needle safety cover 240 is attached to the puncture needle base 220 but can be moved forward and backward along the puncture needle base 220. The puncture needle safety cover 240 further comprises a bladder puncture needle 235, a cover arm 245, and a safety cover cap 280 with a cap post 285.

The bladder puncture needle 235 is disposed in the cover arm 245 with an exposed end of the bladder puncture needle 235 extends into a section of the puncture needle base 220 when the puncture needle safety cover 240 is retracted or moved backward.

A bladder 260 filled with color fluid is housed in an area of the puncture needle base 220 isolated from the IV reservoir 226.

Prior to use the puncture needle safety cover 240 of the IV bag puncture needle assembly 200 is positioned as shown in FIGS. 2E and 2K. Bottom edges of the puncture needle safety cover 240 are positioned in the front holder 222 which holds the puncture needle safety cover 240 in the proper position during shipment and prior to use.

For use, the puncture needle safety cover 240 is retracted or moved backward along the puncture needle base 220 and the bottom edges of the puncture needle safety cover 240 exit the front holder. When the base tip 221 contacts the cap post 285 the safety cover cap 280 is automatically opened and the IV bag puncture needle 230 is exposed through an opening in the puncture needle safety cover 240. The puncture needle safety cover 240 is moved in a backward direction until the bottom edges of the puncture needle safety cover 240 enter the rear lock 224 of the IV bag puncture needle base 240. The rear lock 224 holds the bottom edge and prevents accidental forward movement of the puncture needle safety cover 240 during use as shown in FIGS. 2F and 2M.

After use, the rear lock 224 is depressed thereby releasing the bottom edges and the puncture needle safety cover 240 is moved forward along the puncture needle base 220. After the bottom edges of the puncture needle safety cover 240 enter the front holder 222 the puncture needle safety cover 240 is moved even more forward and the bottom edges enter the front lock 223 as shown in FIG. 2G. The front lock 223 holds the bottom edges of the puncture needle safety cover 240 and prevents backward movement of the puncture needle safety cover 240.

The IV bag puncture needle assembly 200 of the present invention further provides status indicators or used indicator to indicate if the IV bag puncture needle assembly 200 is in use or has been previously used. Prior to use, the position of the puncture needle safety cover 240 is a shown FIG. 2J and the indicators are not visible. During use or when the IV bag puncture needle assembly 200 has been retracted an indicator opening 290 in the IV bag puncture needle assembly 200 allows the using indicator 291 to be visible as shown in FIG. 2I. After use, when the IV bag puncture needle assembly 200 has been moved into the front lock 223 the used indicator 292 is visible as shown in FIG. 2H.

When the puncture needle safety cover 240 is moved backward the bladder puncture needle 235 travels deeper into the puncture needle base 220 until it reaches the bladder 260 filled with color fluid that is disposed in a compartment of the puncture needle base 220 isolated from the IV reservoir 226. The sharp tip of the puncture needle 235 penetrates the bladder 260 and the colored fluid begins to flow into a color reservoir 265 below the bladder 260. The color fluid flows out of the color reservoir 265 and through the puncture needle used line outlet 228 of the puncture needle base 220 and into a used indicator line 120 connected to the puncture needle used line outlet 228.

A spring 299 disposed between the cover arm 245 and the puncture needle base 220 provides tension to maintain the puncture needle safety cover 240 in the rear lock 224.

After the puncture needle safety cover 240 has been moved backward and positioned in the rear lock 224, the puncture needle 230 is pressed against and penetrates the IV bag and the base tip 221 enters the IV bag. The fluid in the IV bag flows through the IV bag puncture needle 230 and into the IV reservoir 226. The fluid continues flowing through the puncture needle IV outlet 227 and into an IV infusion line 110 connected to the puncture needle IV outlet 227.

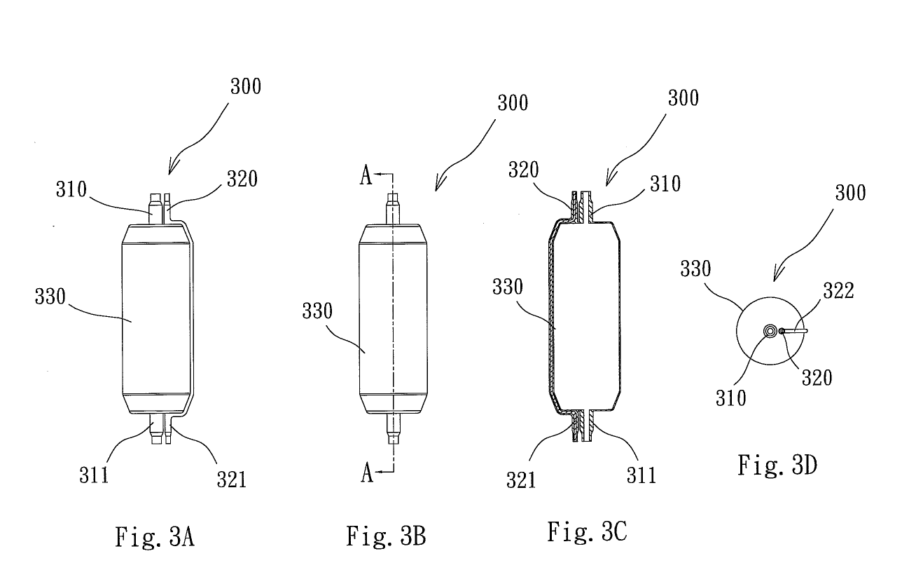

Refer to FIGS. 3A-3D, which are drawings illustrating a drip chamber of a single use intravenous therapy administering device with needle safety covers according to an embodiment of the present invention.

The drip chamber 300 allows for a small quantity of medical fluid to be temporarily stored in order to ensure that a continuous flow of medical fluid reaches the IV needle assembly 500. The use of the drip chamber 300 prevents air entering the blood stream and also allows for an estimation of flow rate.

The drip chamber 300 comprises a drip chamber IV line inlet 310, a drip chamber IV line outlet 311, a drip chamber used line inlet 320, a drip chamber used line outlet 321, a drip chamber used line 322, and a drip chamber body 330.

The drip chamber 300 is connected to the IV bag puncture needle assembly (200) by an IV infusion line (110). One end of the IV infusion line is attached to the drip chamber IV line inlet 310 and the other end of the IV infusion line is attached to the puncture needle IV outlet (227).

Additionally, the drip chamber 300 is connected to the IV bag puncture needle assembly (200) by a used indicator line (120). One end of the used indicator line is attached to the drip chamber used line inlet 320 and the other end of the used indicator line is attached to the puncture needle used line outlet (228).

In application, the IV fluid exiting the puncture needle IV outlet flows through the IV infusion line, into the drip chamber IV line inlet 310, into the drip chamber body 330, and exits the drip chamber 300 from the drip chamber IV line outlet 311. The colored fluid exiting the puncture needle used line outlet flows through the used indicator line 120, into the drip chamber used line inlet 320, through the drip chamber used line 322, and exits from the drip chamber used line outlet 321.

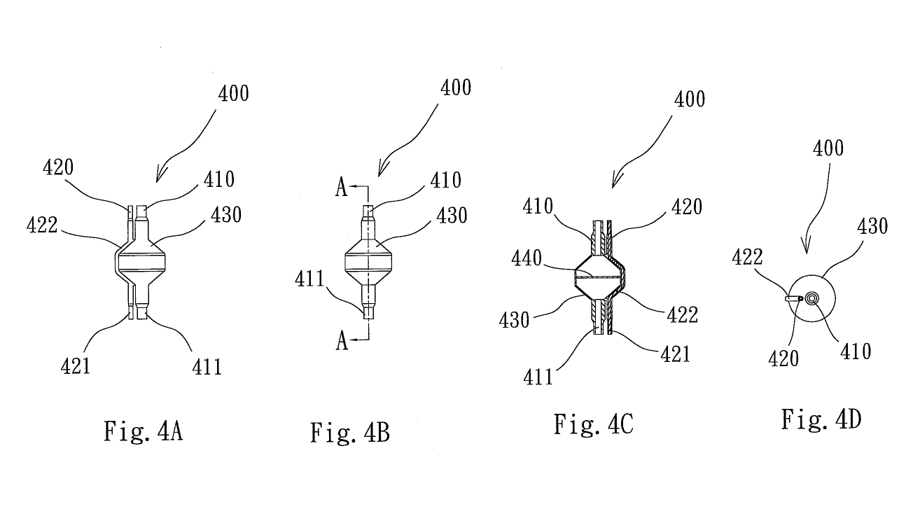

Refer to FIGS. 4A-4D, which are drawings illustrating a fluid filter of a single use intravenous therapy administering device with needle safety covers according to an embodiment of the present invention.

The IV fluid filter 400 filters the IV fluid prior to entering the patient's vein. The IV fluid filter 400 comprises a fluid filter IV line inlet 410, a fluid filter IV line outlet 411, a fluid filter used line inlet 420, a fluid filter used line outlet 421, a fluid filter used line 422, an IV fluid filter body 430, and an IV fluid filter membrane filter 440.

The IV fluid filter 400 is connected to the drip chamber (300) by an IV infusion line (110). One end of the IV infusion line is attached to the fluid filter IV line inlet 410 and the other end of the IV infusion line is attached to the drip chamber IV line outlet 311.

Additionally, the IV fluid filter 400 is connected to the drip chamber by a used indicator line (120). One end of the used indicator line is attached to the fluid filter used line inlet 420 and the other end of the used indicator line is attached to the drip chamber used line outlet (321).

In application, the IV fluid exiting the drip chamber IV outlet flows through the IV infusion line, into the fluid filter IV line inlet 410, into the fluid filter body 430, passes through the IV fluid filter membrane filter 440, and exits the IV fluid filter 400 from the fluid filter IV line outlet 411. The colored fluid exiting the drip chamber used line outlet flows through the used indicator line 120, into the fluid filter used line inlet 420, through the fluid filter used line 422, and exits from the fluid filter used line outlet 421.

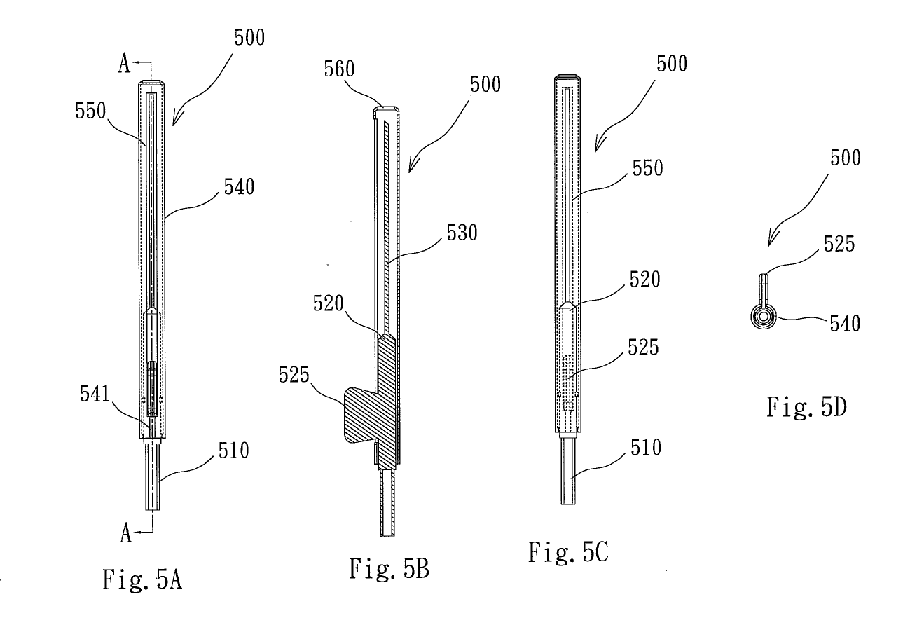

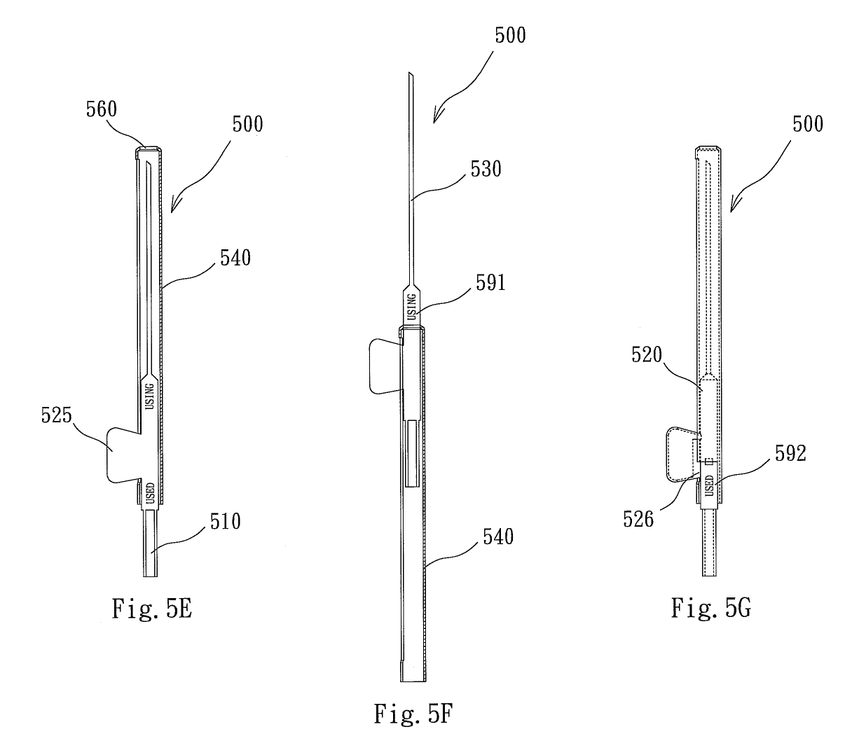

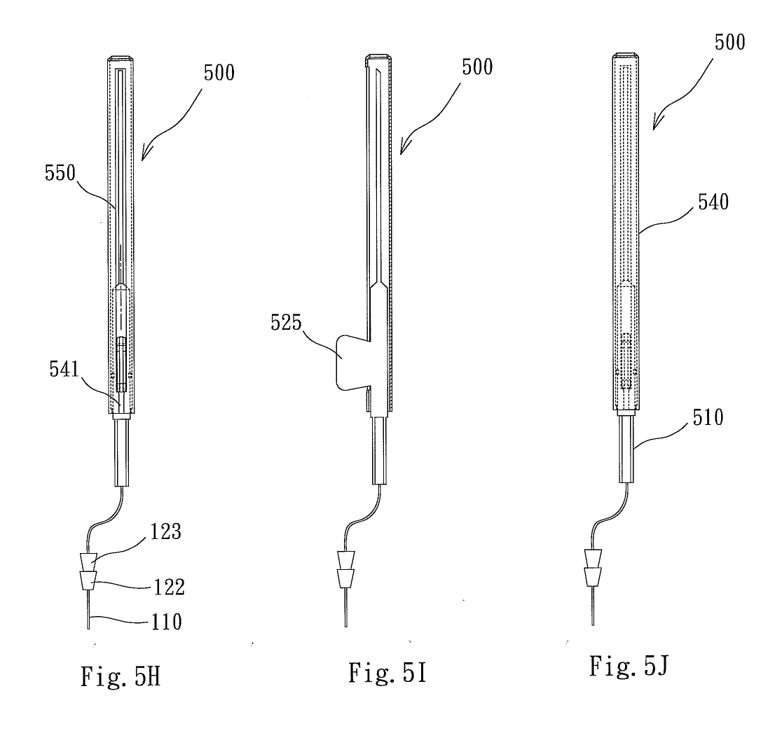

Refer to FIGS. 5A-5J, which are drawings illustrating an intravenous needle assembly of a single use intravenous therapy administering device with needle safety covers according to embodiments of the present invention.

The IV needle assembly 500 allows the IV fluid to flow from the IV infusion line 110 and into a patient's vein. The IV needle assembly 500 comprises an IV needle base 520, an IV needle 530, and an IV needle safety cover 540.

One end of the IV needle 530 is disposed inside the IV needle base 520 with the other sharp end protruding from the IV needle base 520. The IV needle base 520 further comprises an IV line needle assembly inlet 510 for connecting with the IV infusion line 110 and an IV needle base grip 525 to facilitate holding of the IV needle assembly 500.

The IV needle safety cover 540 surrounds the IV needle 530 to prevent accidental contact with medical personnel or patients. The IV needle safety cover 540 further comprises an IV needle safety cover lock 541, an IV needle safety cover channel 550, and an IV needle safety cover end opening 560.

In use, the IV needle safety cover 540 is retracted or pulled backward along the IV needle base 520 and the IV needle 530 is exposed through the IV needle safety cover end opening 560. While being retracted the IV needle safety cover channel 550 and the IV needle base grip 525 cooperate to hold the IV needle safety cover 540 in alignment. After use, the IV needle safety cover 540 is moved forward to cover the IV needle 530. By continuing to move the IV needle safety cover 540 forward passed its original position on the IV needle base 520, the IV needle safety cover lock 541 disposed on the end of the IV needle safety cover 540 slides into an IV needle grasp lock 526 on the IV needle base 520. Once the IV needle safety cover lock 541 enters the IV needle grasp lock 526 the IV needle safety cover 540 is held in a locked position.

The IV needle base further comprises an IV needle using indicator 591 and an IV needle used indicator 592. Prior to use, the IV needle using indicator 591 and the IV needle used indicator 592 are covered by the IV needle safety cover 540 and are not visible as shown in FIG. 5E. During use, when the IV needle safety cover 540 is retracted the IV needle using indicator 591 is visible and identifies the IV needle assembly 500 as being in use or using as shown in FIG. 5F. After use, when the IV needle safety cover 540 is moved forward and into the IV needle safety cover lock 541 the IV needle used indicator 592 becomes visible and identifies the IV needle assembly 500 as used as shown in FIG. 5G. The IV needle used indicator 592 allows medical personnel to easily determine that the IV needle assembly 500 has been previously used and prevents inadvertent re-use.

In application, the IV fluid exiting the fluid filter IV outlet flows through the IV infusion line 110, into the IV needle assembly inlet 510, through the IV needle 530, and into the patient's vein that the IV needle 530 has been inserted into.

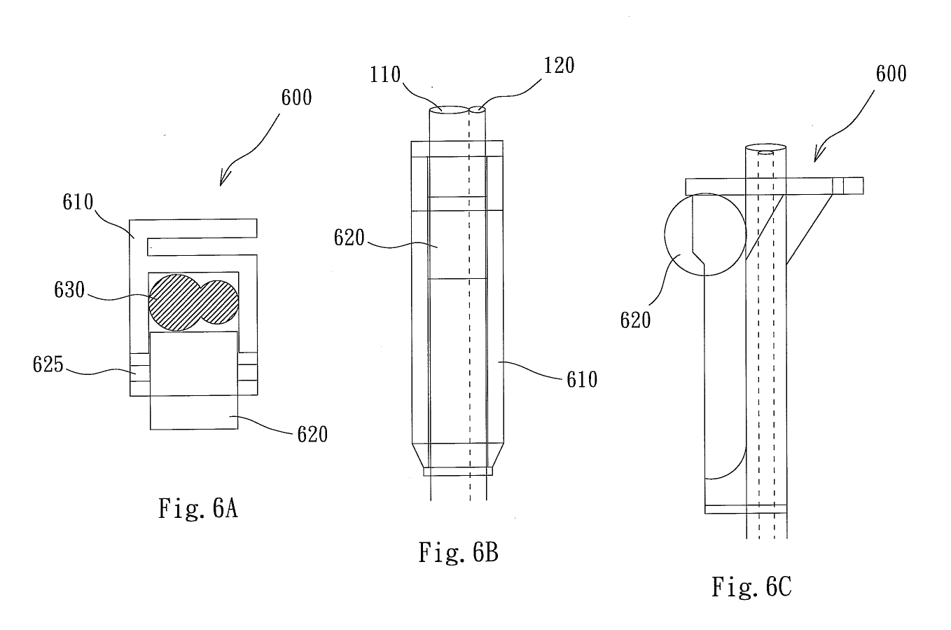

Refer to FIGS. 6A-6C, which are drawings illustrating a flow regulator of a single use intravenous therapy administering device with needle safety covers according to an embodiment of the present invention.

Disposed between the drip chamber (300) and the IV fluid filter (400) is a flow regulator 600 for controlling or regulating the flow of the IV fluid through the IV infusion line (100).

The flow regulator 600 comprises a regulator body 610 and a roller 620. The roller 620 further comprises a roller axle 625. The roller axle 625 travels in a channel groove of the regulator body 610 allowing the roller 620 to move forward and backward while holding the roller 620 in the regulator body 610. As the roller 620 is moved the roller axle 625 travels in the channel groove and the roller 620 slowly compresses the IV infusion line 110 and slows the flow of the IV fluid. When the roller 620 is moved in the opposite direction the roller releases the pressure on the IV infusion line 110 and the flow of the IV fluid returns to the original flow capacity.

The regulator body 610 further comprises a keyed opening 630. The keyed opening 630 is formed in the shape of the IV infusion line 110 and the used indicator line 120. When the IV infusion line 110 and the used indicator line 120 are positioned in the keyed opening 630, the IV infusion line 110 and the used indicator line 120 can not be rotated or turned in the regulator body 610. The keyed opening 630 ensures that the roller 620 does not compress the used indicator line 120 when the roller 620 is moved to compress the IV infusion line 110 which could happen if the IV infusion line 110 and the used indicator line 120 were allowed to be turned in the regulator body 610.

Since the flow regulator 600 surrounds but is not attached to the IV infusion line 110 and the used indicator line 120, the flow regulator's location can be adjusted by sliding the flow regulator along the two lines 110 120.

It will be apparent to those skilled in the art that various modifications and variations can be made to the present invention without departing from the scope or spirit of the invention. In view of the foregoing, it is intended that the present invention cover modifications and variations of this invention provided they fall within the scope of the invention and its equivalent.

What is claimed is:

1. An intravenous therapy administering device comprising:
an intravenous bag puncture needle assembly comprising an intravenous bag puncture needle and a puncture needle safety cover surrounding the intravenous bag puncture needle;
an intravenous needle assembly;
an infusion line connecting the intravenous bag puncture needle assembly and the intravenous needle assembly; and
a used indicator line parallel to the infusion line connected to the intravenous bag puncture needle assembly.

2. The intravenous therapy administering device of claim 1, further comprising:
a drip chamber; and
a fluid filter, the infusion line connecting the intravenous bag puncture needle assembly and the drip chamber, the infusion line connecting the drip chamber and the fluid filter, the infusion line connecting the fluid filter and the intravenous needle assembly.

3. The intravenous therapy administering device of claim 1, the intravenous puncture needle assembly further comprising:
a bladder containing colored fluid; and
a bladder puncture needle disposed in the puncture needle safety cover, the bladder puncture needle penetrating the bladder when the puncture needle safety cover is retracted.

4. An intravenous therapy administering device comprising:
an intravenous bag puncture needle assembly comprising an intravenous bag puncture needle and a puncture needle safety cover surrounding the intravenous bag puncture needle;
a drip chamber;
a fluid filter;
an intravenous needle assembly;
an infusion line connecting the intravenous bag puncture needle assembly and the drip chamber, the infusion line connecting the drip chamber and the fluid filter, the infusion line connecting the fluid filter and the intravenous needle assembly; and
a used indicator line parallel to the infusion line connected to the intravenous bag puncture needle assembly.

5. The intravenous therapy administering device of claim 4, further comprising:
a flow regulator disposed between the drip chamber and the fluid filter for controlling flow of intravenous fluid through the infusion line.

6. The intravenous therapy administering device of claim 4, the drip chamber comprising:
a drip chamber infusion line inlet connected by the infusion line to the intravenous bag puncture needle assembly;
a drip chamber infusion line outlet connected by the infusion line to the fluid filter;
a drip chamber used line inlet connected by the used indicator line to the intravenous bag puncture needle assembly;
a drip chamber used line outlet connected by the used indicator line to the fluid filter;
a drip chamber body disposed between the drip chamber infusion line inlet and the drip chamber infusion line outlet; and
a drip chamber used line on an outside of the drip chamber body connecting the drip chamber used line inlet and the drip chamber used line outlet.

7. The intravenous therapy administering device of claim 4, the fluid filter comprising:
a fluid filter infusion line inlet connected by the infusion line to the drip chamber;
a fluid filter infusion line outlet connected by the infusion line to the fluid intravenous needle assembly;
a fluid filter used line inlet connected by the used indicator line to the drip chamber;
a fluid filter used line outlet;
a fluid filter body disposed between the fluid filter infusion line inlet and the fluid filter infusion line outlet;
a fluid filter membrane filter disposed inside the fluid filter body; and
a fluid filter used line on an outside of the fluid filter body connecting the fluid filter used line inlet and the fluid filter used line outlet.

8. The intravenous therapy administering device of claim 4, the intravenous puncture needle assembly further comprising:
a bladder containing colored fluid.

9. The intravenous therapy administering device of claim 8, the intravenous puncture needle assembly further comprising:
a bladder puncture needle disposed in the puncture needle safety cover, the bladder puncture needle penetrating the bladder when the puncture needle safety cover is retracted.

10. The intravenous therapy administering device of claim 9, wherein the colored fluid flows into the used indicator line when the bladder has been punctured.

11. The intravenous therapy administering device of claim 4, the intravenous puncture needle assembly further comprising:
a puncture needle used indicator for indicating that the intravenous bag puncture needle has been used.

12. The intravenous therapy administering device of claim 4, the intravenous puncture needle assembly further comprising:
a front holder for holding the puncture needle safety cover in place prior to use.

13. The intravenous therapy administering device of claim 4, the intravenous puncture needle assembly further comprising:
a front lock for holding the puncture needle safety cover in place after use.

14. The intravenous therapy administering device of claim 4, the intravenous puncture needle assembly further comprising:
an air filter disposed in a puncture needle base, the air filter comprising an air hole and an air filter membrane.

15. The intravenous therapy administering device of claim 4, the puncture needle safety cover further comprising:
a safety cover cap disposed in a top surface of the puncture needle safety cover for covering a opening in the puncture needle safety cover over the intravenous bag puncture needle.

16. The intravenous therapy administering device of claim 14, the puncture needle safety cover further comprising:
an air filter cap disposed in a side of the puncture needle safety cover for covering the air hole of the air filter.

17. The intravenous therapy administering device of claim 4, the intravenous needle assembly comprising:
an intravenous needle base;
an intravenous needle partially surrounded by the intravenous needle base; and
an intravenous needle safety cover.

18. The intravenous therapy administering device of claim 4, the intravenous needle assembly further comprising:
an intravenous needle used indicator disposed on the intravenous needle base for indicating that the intravenous needle has been used.

19. An intravenous therapy administering device comprising:
an infusion line;
a used indicator line, the used indicator line parallel to the infusion line and connected to an intravenous bag puncture needle assembly;
the intravenous bag puncture needle assembly comprising:
an intravenous bag puncture needle;
a puncture needle base;
a puncture needle safety cover surrounding the intravenous bag puncture needle;
a bladder containing colored fluid;
a bladder puncture needle disposed in the puncture needle safety cover, the bladder puncture needle penetrating the bladder when the puncture needle safety cover is retracted;
an air filter disposed in a side of the puncture needle base, the air filter comprising an air hole and an air filter membrane;
an air filter cap disposed on the puncture needle safety cover for covering the air hole of the air filter; and
a safety cover cap disposed in a top surface of the puncture needle safety cover for covering an opening in the puncture needle safety cover over the intravenous bag puncture needle;
a drip chamber, the drip chamber comprising:
a drip chamber infusion line inlet connected by the infusion line to the intravenous bag puncture needle assembly;
a drip chamber infusion line outlet connected by the infusion line to a fluid filter;
a drip chamber used line inlet connected by the used indicator line to the intravenous bag puncture needle assembly;
a drip chamber used line outlet connected by the used indicator line to the fluid filter;
a drip chamber body disposed between the drip chamber infusion line inlet and the drip chamber infusion line outlet; and
a drip chamber used line on an outside of the drip chamber body connecting the drip chamber used line inlet and the drip chamber used line outlet;
the fluid filter, the fluid filter comprising:
a fluid filter infusion line inlet connected by the infusion line to the drip chamber;
a fluid filter infusion line outlet connected by the infusion line to an intravenous needle assembly;
a fluid filter used line inlet connected by the used indicator line to the drip chamber;
a fluid filter used line outlet;
a fluid filter body disposed between the fluid filter infusion line inlet and the fluid filter infusion line outlet;
a fluid filter membrane filter disposed inside the fluid filter body; and
a fluid filter used line on an outside of the fluid filter body connecting the fluid filter used line inlet and the fluid filter used line outlet;
the intravenous needle assembly, the intravenous needle assembly comprising:
an intravenous needle base;
an intravenous needle partially surrounded by the intravenous needle base; and
an intravenous needle safety cover; and
a flow regulator disposed between the drip chamber and the fluid filter for controlling flow of intravenous fluid through the infusion line.

20. The intravenous therapy administering device of claim 19, the intravenous puncture needle assembly further comprising:
a used indicator for indicating that the intravenous bag puncture needle has been used.
